(19) United States
(12) Patent Application Publication
(10) Pub. No.: US 2006/0173284 A1
(43) Pub. Date: Aug. 3, 2006

(54) Radiofrequency coil and catheter for surface nmr imaging and spectroscopy

(76) Inventors: Jerome L. Ackerman, Newton, MA (US); Van J. Wedeen, Somerville, MA (US)

Correspondence Address: NUTTER MCCLENNEN & FISH LLP, WORLD TRADE CENTER WEST, 155 SEAPORT BOULEVARD, BOSTON, MA, 02210-2604, US

(21) Appl. No.: 10/532,156

(22) PCT Filed: Oct. 21, 2003
(86) PCT No.: PCT/US03/33316

§ 371 Date: Nov. 7, 2005

Related U.S. Application Data
(60) Provisional application No. 60/419,987, filed on Oct. 21, 2002.

(51) Int. Cl. A61B 5/05

(52) U.S. Cl. 600/422

(57) ABSTRACT

In one aspect, the present invention provides a cylindrical meanderline coil that can significantly improve the performance and usefulness of nuclear magnetic resonance (NMR) catheter radiofrequency (RF) coils by shaping the spatial dimensions of the volume of excitation and reception of signal. This can provide improved accuracy in defining the volume of excitation and reception of the subject or specimen, and increase the signal to noise ratio of a received signal. In another aspect, the invention provides an intravascular catheter having a coil at its tip for generating and/or detecting magnetic excitations. A preamplifer coupled to the catheter in proximity of the coil allows amplifying signals generated and/or detected by the coil. Although in one application, a coil and/or a catheter of the invention can be employed, for example, for MR spectroscopy or imaging of biological tissue, such as atherosclerotic plaques arterial walls in the human body, the invention provides similar advantages in any situation where a magnetic resonance or other magnetic induction signal is to be received from a thin cylindrical shell or sector of a cylindrical shell.

### RELATED APPLICATIONS

This application claims priority to provisional application No. 60/419,987 entitled “Radiofrequency coil and catheter for surface NMR imaging and spectroscopy,” filed on Oct. 21, 2002.

The Government has rights in this invention pursuant to Cooperative Agreement Number DAMD17-02-2-0006.

### BACKGROUND OF THE INVENTION

The present invention relates generally to devices for magnetic resonance (MR) spectroscopy and/or imaging, and more particularly, to an enhanced coil design and a catheter suitable for use in MR spectroscopy and/or imaging.

In magnetic resonance (MR) scanners, the nuclear spins of a subject are aligned by an intense static (constant) magnet field B₀, and perturbed by an oscillating (typically radiofrequency) magnetic field B₁ (perpendicular to B₀) generated by current flowing in one or more inductive structures, usually referred to as coils or RF coils. Following the perturbation, the nuclear spins emit oscillatory magnetic fields that are converted to oscillatory electrical signals by either the same RF coil or coils, or by a different coil or set of coils. These nuclear signals are detected by the MR scanners and converted to NMR spectra, which reveal chemical composition, or nuclear magnetic resonance images of the subject. The corresponding methodologies are generally known as magnetic resonance spectroscopy (MRS) and magnetic resonance imaging (MRI), respectively.

Normally, an RF coil forms an inductive component of a tuned resonant electrical circuit. It is the oscillatory magnetic field of the coil that excites the nuclear spins when an oscillatory electrical current flows through the electrical conductors of the coil. The spatial pattern of the magnetic field, i.e., the intensity and direction of the magnetic field at every point in space, generated by the coil determines the spatial pattern of excitation, and is itself determined by the spatial arrangement of the electrical conductors of the coil.

In the most general situation, as shown in FIG. 1A, the subject to be scanned, for example, a test sample, an animal, a person, or a part—head, limb, etc—of an animal or person, is placed completely within the RF coil that can generate magnetic fields for exciting selected spins of the subject and can also function as a detector for transducing the magnetic fields generated by the spins into electrical signals. Performance of the coil, both as a generator and detector of magnetic fields, is related, among many other parameters, to a quantity known as filling factor η that is defined roughly as a fraction of the coil volume that is occupied by the volume of interest. It is of critical importance to maximize the filling factor, which in the case of a coil that completely encompasses the subject, is achieved by making the coil as small as possible while still being capable of completely encompassing the subject. Such coils are often referred to as volume coils. These may be constructed as simple solenoids, for example, a simple helically wound coil of wire, or as Helmholtz coils, for example, a pair of identical coaxial coils lying on parallel planes spaced apart by a distance equal to the coil radius, or more complex structures such as birdcage coils.

When the volume of interest is a relatively small part of the entire subject, and located in the vicinity of the subject's surface, an improvement in the filling factor may be achieved by employing a small RF coil placed against the subject, as shown in FIG. 1B. This type of coil is known as a local or surface coil. An example of the use of a surface coil is the placement of an RF coil formed of a single loop of wire against a person's chest to interrogate the heart by MRS or MRI. Although the oscillatory magnetic field B₁ generated by such a surface coil is spatially highly nonuniform compared with that generated by a solenoid, the improvement in filling factor, and therefore in signal to noise ratio, typically vastly outweighs the problems created by the nonuniform field. Despite some of their disadvantages, surface coils confer substantial advantages over volume coils when the volume of interest is much smaller than the volume of the subject, provided that the coil can be placed in a suitable location, and provided that appropriate adjustments are made in the operation of the scanner. Generally, the coil's dimensions roughly determine its volume of useful sensitivity. For example, in the case of a simple circular wire loop surface coil, the sensitive volume is roughly the spherical volume defined by the circumference of the loop. More precisely, the sensitivity is maximal at the center of the loop, and falls off smoothly as the distance from the plane of the loop increases. For optimal performance, the dimensions of the surface coil should be roughly comparable to the dimensions of the volume of interest.

When the volume of interest is an atherosclerotic plaque located in the wall of a coronary artery, even an externally placed surface coil having dimensions on the order of the plaque dimensions provides an extremely low filling factor because the plaque is likely to be far away from such a surface coil. If the surface coil is sized so that the plaque is well within the coil's sensitive volume, the surface coil diameter will need to be much larger than the plaque diameter, thus yielding a poor filling factor.

The shape and intensity distribution of the reception volume of the coil may be further tailored with specific arrangements of the electrical conductors forming the coil. This may be for enhancing the uniformity of the excitation throughout a volume of interest, or for more sharply defining the shape of the volume, or both. For example, the excitation volume of a solenoid coil is usually considered to be defined by the geometric volume of the cylinder on which the solenoid is wound. However, the magnetic field produced by the solenoid, which defines both its reception and excitation volumes, actually extends throughout all space. The field is strongest, and mostly confined, within the volume of the cylinder, but is present to some extent everywhere. The solenoid produces a moderately uniform field intensity and direction within its cylindrical volume. The intensity of the field on the solenoid axis varies by about a factor of two from the center of the cylinder to its ends, and the direction of the on-axis field is parallel to the axis. At large distances from the solenoid, the field is approximately dipolar in shape and varies approximately with the inverse third power of the distance.

A surface coil formed of a single loop of wire, which may be considered to be a solenoid of approximately zero length, has a much more drastic variation of field intensity than does a solenoid of finite length, with the on-axis intensity falling off roughly as the inverse third power of the distance from the plane of the loop. Alternatively, birdcage coils are designed to produce a highly uniform field within the geometrical volume, with the field direction perpendicular to the birdcage cylinder axis.

Another known RF coil design is a flat coil that includes a planar rectangular array of conductors interconnected so that a current flowing from one end of the coil to the other end has opposite directions in adjacent conductors.

Another type of RF coil is known as the shorted line, or slotted line, or transmission line, coil. A coaxial transmission line (coaxial cable) may be shorted at one end to create standing electromagnetic waves within the line. The line therefore becomes a resonant structure that can be tuned and matched to the characteristic impedance (typically, but not necessarily, 50 ohms) of the scanner's receiver, similar to a conventional RF coil. At an appropriate distance from the short, an opening, or aperture, is cut in the shield to expose the central conductor. The magnetic field within the line leaks out of the opening and permits the slotted line to be used as an intravascular coil for MR scanning. The cross sectional shape of the field is approximately defined by the length and width of the aperture. The intensity of the field in the vicinity of the center conductor falls roughly as the reciprocal of the distance from the center conductor, and faster with increasing distance.

Conventional intravascular coils often take the form of simple loops of wire. The geometrically simplest type of an intravascular coil is essentially a bare wire (such as the guide wire of a catheter), or a length of small diameter coaxial cable with a length of the shield removed from the end (the “loopless antenna”). The current return path of a loopless antenna is via the capacitance between the bare wire and the shield of the coaxial cable. Although this is a rather poor coil compared to a true loop or solenoid, it has the advantage that it can be made very small so that it fits easily into blood vessels, and it still gives a strongly improved filling factor compared to any coil that is placed external to the body. It has, however, the disadvantage that its volume of sensitivity is concentrated near the exposed wire. The loopless antenna, and guidewires used as coils, are therefore most sensitive to the blood (which is usually not of interest in intravascular MR scanning), and less sensitive to vessel walls (which are usually of most interest in intravascular MR scanning). An additional problem caused by this greater sensitivity to blood rather than vessel walls is that the blood MR signal, being enhanced relative to the vessel wall signal, tends to dominate and obscure signals from the vessel wall. A third problem associated with such conventional intravascular coils is that motion artifacts due to the flow of the blood also tend to obscure signals from the vessel wall. A fourth problem caused by the increased sensitivity to volumes that are not of interest is that electrical noise is unnecessarily detected from these volumes of tissue which cannot be removed from the image or spectrum, thereby reducing the signal-to-noise ratio.

Normally, the coils employed for MR spectroscopy and imaging form the inductive components of tuned resonant electrical circuits. For effective use, the circuits must be accurately tuned to the Larmor (precession) frequency of the nuclear spins that are excited and detected. The electrical cables connecting the coils to other components of the tuned resonant circuit can introduce signal loss that can adversely affect the signal to noise ratio of the detected signal. For example, because of the confined space within a blood vessel, a coaxial cable utilized to connect a coil to an external scanner is typically of small diameter and therefore of high attenuation, which causes loss of signal to noise ratio. Some or all of the tuning capacitors of some conventional intravascular coils are fixed in value so that they can be placed near the coil. These values are typically selected as a compromise among the full range of values that would be needed to all possible tuning conditions. This, however, limits the available tuning conditions of the coil, which can in turn degrade the performance of the coil. For example, intravascular coils, when placed in blood vessels, are in constant motion because of the heart beat, the pulsatile flow of blood, and other voluntary and/or involuntary motions of the subject. Such motions cause the optimal tuning conditions to be continuously changing during a scan, thus requiring a continuous adjustment of the tuning capacitors if optimal tuning is desired. The optimal tuning conditions may also change as the catheter containing the coil is advanced through a vessel or is pulled back. Hence, the inability to adjust the capacitance can result in operating the intravascular coil under conditions of highly compromised tuning, which in turn can result in a low signal-to-noise ratio.

Alternatively, some or all of the tuning components of an intravascular coil can be placed outside the body of the subject, and hence at a substantial distance from the coil, so that the capacitance can be adjusted. This approach, however, can result in a severe loss of signal-to-noise ratio because of the high attenuation of a small diameter cable that needs to be employed to connect the coil to the external capacitors.

Thus, there is a need for a coil for use in MR spectroscopy and/or imaging that can allow scanning biological tissue such as arterial plaques, blood clots, or the brain cortex.

There is also a need for such a coil that can provide enhanced filling factors for imaging curved surfaces while reducing its sensitivity to materials disposed outside these surfaces, particularly when utilized as an intravascular coil for MR spectroscopy or imaging of arterial plaques or blood clots, or when used externally to scan the brain cortex.

There is further a need for an intravascular catheter having a coil for performing MR spectroscopy and/or imaging that can be continuously tuned while disposed in a blood vessel.

Moreover, there is a need for an intravascular catheter having a coil for MR spectroscopy in which signals detected by the coil can be amplified and transmitted to an external circuitry with minimal attenuation.

### SUMMARY OF THE INVENTION

In one aspect, the present invention provides a coil for transmitting and detecting magnetic excitations. A coil of the invention can include a meanderline, also referred to as zigzag or serpentine, conductive structure having a plurality of conductive segments that form a substantially cylindrical profile to generate non-vanishing magnetic fields, in response to a current flow through the coil, in a substantially annular region surrounding the conductive segments, and substantially vanishing magnetic fields outside the annular region. For example, the substantially vanishing magnetic field can be weaker than the average magnetic field generated in the annular region by about 10 dB, or preferably by about 20 dB, or more preferably by about 40 dB or more. Most preferably, the magnetic field completely vanishes outside the annular region.

In a related aspect, a meanderline conductive structure of a coil of the invention includes an input lead and an output lead, and each conductive segment forming a portion of the conductive structure is composed of at least a pair of elongated conductors disposed substantially parallel to one another, and spaced apart by a selected distance, such that a current flow through the coil, for example, from the input lead to the output lead, results in opposite current directions in each conductor of the pair. In this manner, a non-vanishing magnetic field distribution can be generated in a generally annular region surrounding the conductive segments while the magnetic field falls off to very low values outside the annular region. For example, when the coil exhibits a substantially cylindrical profile, the non-vanishing magnetic field distribution can be in the form of a cylindrical shell surrounding the coil's conductive segments with the magnetic field strength decreasing rapidly to vanishing values beyond the shell's inner and outer boundaries.

The annular region typically has a width that is commensurate, i.e., it is of the order of, the spacing between the pair of elongated conductors of each conductive segment of the coil. A width of the annular region can be defined, for example, as a distance between inner and outer boundaries of the region, where each boundary represents a location at which the magnetic field strength is reduced by a selected amount, e.g., by 1/e, relative to its values at the center of the annular region.

In other aspects, capacitors can be coupled to the coil, for example, distributed along the coil, for providing tuning and/or impedance matching of the coil. The capacitance can be in the form of discrete devices, or can be in a continuously distributed form as in the capacitance between the coil and a ground plane. Varactor diodes can be utilized as adjustable capacitors whose capacitance can be varied by adjusting one or more DC voltages applied thereto.

A coil of the invention can be utilized for RF excitation, detection, or both. Thus, the terms “excitation” and “volume of excitation” can also be understood to refer to reception and volume of reception, respectively.

In another aspect, the present invention provides a coil assembly, formed of at least a pair of conductive coils, for radiofrequency (RF) quadrature operation. Each conductive coil can include an input terminal, an output terminal, and a plurality of conductive segments that extend from the input terminal to the output terminal. Each conductive segment includes two elongated conductors that are disposed substantially parallel to one another such that a flow of a current from the input terminal to the output terminal results in opposite current directions in the conductors. The conductors are disposed in proximity of one another such that application of two voltage signals, having substantially equal amplitudes and a phase difference of about 90 degrees, each across one of the coils, causes currents in the coils so as to generate a circularly polarized RF magnetic field.

In a related aspect, in the coil assembly described above, the coils are disposed relative to one another such that the conductors of each conductive segment of one coil are substantially perpendicular to the conductors of a corresponding conductive segment of the other coil. The coils utilized in the coil assembly can have a variety of different profiles. For example, each coil can be flat, or have a cylindrical profile. Alternatively, the profile of both or at least one of the coils can correspond to a sector of a cylinder.

In another aspect, the present invention provides a medical catheter that includes a flexible body extending from a proximal end to a distal end, and a coil coupled to the flexible body in proximity of the distal end for generating and detecting magnetic signals. A miniature amplifier is coupled to the catheter in proximity of the coil, and is electrically connected thereto, in order to amplify magnetic signals generated or detected by the coil.

In a related aspect, in a medical catheter according to the teachings of the invention as described above, the coil can include a meanderline conductive structure having a plurality of segments that form a substantially cylindrical profile. The conductive segments are configured such that the coil generates, in response to a current flow therethrough, a non-vanishing magnetic field in a region in proximity of the conductive segments and a substantially vanishing magnetic field in a region removed from the proximity of the conductive segments.

In another aspect, the catheter includes a flexible body, formed preferably of a biocompatible material, that is sized to allow navigating the catheter through a patient's circulatory system, for example, a patient's artery. Further, capacitive and/or inductive elements can be coupled to the coil to allow tuning it to a selected frequency. Moreover, an elongated conductor, which extends from the proximal end of the catheter to its distal end, can be employed to transmit excitation signals from an external circuitry to the coil and/or transmit signals detected by the coil, for example, signals emitted by nuclear spins in response to excitation by the coil, to an external circuitry.

In another aspect, the invention provides a catheter that can be utilized in two operational modes, in one of which an extended length of a vessel can be imaged, and in the other, a smaller portion of the vessel wall can be imaged. Such a catheter can include an elongate conductor that extends along a portion thereof, for example, from its proximal end to its distal end, and a coil according to the teachings of the invention coupled to its distal end. An external circuitry coupled to the elongate conductor can be employed to tune the elongate conductor for imaging an extended length of the vessel, and another external circuitry coupled to the coil can be utilized to tune the coil for imaging a portion of the vessel in proximity of the coil.

In yet another aspect, the present invention provides a method for magnetic resonance imaging of at least a portion of a plaque disposed on an inner wall of a patient's artery by disposing a coil having a substantially cylindrical profile, formed of a plurality of conductive segments, in the artery in proximity of the plaque. The conductive segments of the coil are configured such that a current flow through the coil generates a substantially vanishing magnetic field in a region within the cylindrical profile through which blood flows, and a non-vanishing magnetic field in an annular region in proximity of the conductive segments extending into at least a portion of the plaque. A static magnetic field is applied to the plaque in order to polarize selected atomic nuclei of the constituents of the plaque. Further, a time-varying signal is applied to the coil, or to another coil, e.g., a coil disposed in a scanner, so as to generate a time-varying magnetic field in the annular region to excite the polarized nuclei. The coil is then employed to detect radiation emitted by the nuclei in response to the excitation. The detected signal can be analyzed, for example, by an external system, in order to ascertain constituents of the plaque. It should be appreciated that an intravascular coil according to the teachings of the invention can be utilized for transmitting and detecting magnetic signals, or only for detecting magnetic signals. When the coil is utilized for only detecting magnetic signals, another coil, e.g., a coil disposed in a magnetic resonance scanner, can be employed for transmitting magnetic excitations, for example, for exciting selected nuclear spins.

Further understanding of the invention can be obtained by reference to the following description in conjunction with associated drawings described briefly below.

### BRIEF DESCRIPTION OF THE DRAWINGS

FIGS. 1A and 1B schematically illustrate, respectively, the use of volume and surface coils for performing magnetic resonance spectroscopy and/or imaging,

FIG. 2 schematically illustrates a cylindrical meanderline coil according to the teachings of the invention,

FIG. 3 schematically illustrates a photolithography mask having a meanderline structure that can be utilized in one step of a method for constructing a cylindrical meanderline coil of the invention,

FIG. 4 depicts transverse and axial views of a simulated magnetic field distribution generated by a cylindrical meanderline coil according to the teachings of the invention,

FIG. 5 schematically illustrates a cylindrical meanderline coil of the invention having a plurality of capacitors for frequency tuning and/or impedance matching of the coil,

FIG. 6A depicts a meanderline cylindrical coil of the invention formed of six conductors and having a 3 mm cross-sectional diameter,

FIG. 6B illustrates an MR image of water obtained by utilizing the coil of FIG. 6A,

FIG. 7 schematically depicts a meanderline cylindrical coil of the invention in which an electromagnetic shield is incorporated,

FIG. 8 schematically depicts incorporation of matching/tuning capacitors in the coil/shield arrangement of FIG. 7,

FIG. 9 depicts transverse and axial views of a simulated magnetic field distribution generated by a cylindrical meanderline coil of the invention having an electromagnetic shield,

FIG. 10A schematically depicts a coil arrangement according to the teachings of the invention formed of two flat meanderline coils that is suitable for quadrature or double resonance operation,

FIG. 10B schematically depicts a coil arrangement according to the teachings of the invention formed of two coaxially interleaved cylindrical meanderline coils suitable for quadrature or double resonance operation,

FIG. 11A schematically depicts another coil arrangement according to the teachings of the invention suitable for quadrature or double resonance operation that is formed of two flat meanderline coils disposed orthogonal relative to one another,

FIG. 11B schematically depicts another coil arrangement according to the teachings of the invention suitable for quadrature or double resonance operation formed of two cylindrical meanderline coils disposed coaxially relative to one another such that their respective conductors are substantially orthogonal,

FIG. 12 schematically depicts the use of a cylindrical meanderline coil of the invention for performing magnetic resonance spectroscopy or imaging on an arterial plaque or blood clot,

FIG. 13A schematically illustrates a catheter according to the teachings of the invention having a coil in proximity of a distal end thereof and a preamplifier disposed in proximity of the coil and electrically coupled thereto in order to amplify signals detected by the coil,

FIG. 13B schematically illustrates a catheter according to the teachings of the invention having a coil at a distal end thereof in which an electronic package coupled to the coil is placed a few centimeters away from the coil,

FIG. 14A is an exemplary circuit diagram for constructing the preamplifier of FIG. 13A,

FIG. 14B is a circuit diagram for a coil/preamplifier for use in a catheter of the invention having amplification and tuning/matching stages,

FIG. 15A is a circuit diagram illustrating the use of capacitors positioned external to a catheter of the invention for tuning and/or impedance matching,

FIG. 15B is a circuit diagram illustrating disposing tuning/matching capacitors in a catheter of the invention in proximity of coil coupled to a distal end of the catheter,

FIGS. 15C and 16 are circuit diagrams illustrating the use of varactor diodes as adjustable capacitors in a catheter of the invention for tuning and/or impedance matching,

FIG. 16 is a flow chart depicting various steps in an exemplary method according to the teachings of the invention for periodically adjusting tuning of an intravascular coil,

FIG. 17 schematically illustrates the use of a catheter of the invention for MR spectroscopy or imaging of arterial plaques and/or blood clots,

FIG. 18 presents preliminary experimental data demonstrating the tight confinement of the sensitive volume of an exemplary cylindrical meanderline coil according to the teachings of the invention,

FIG. 19 schematically depicts another coil of the invention formed as a conductive tube,

FIG. 20 schematically illustrates a tubular coil according to the teachings of the invention, formed of a plurality of conductive segments,

FIG. 21A depicts an exemplary catheter in accordance with one embodiment of the invention capable of operating in two different modes, operating in a mode in which a meanderline coil coupled at its tip is employed for imaging,

FIG. 21B depicts the catheter of FIG. 21A operating in a different mode in which the entire catheter is employed as a guidewire type interavascular coil for imaging an extended length of a vessel wall, and

FIG. 22 schematically depicts a catheter according to one embodiment of the invention having a cylindrical meanderline coil and a balloon at a distal end thereof.

### DETAILED DESCRIPTION OF THE INVENTION

In one aspect, the present invention provides coils that can be utilized for magnetic resonance imaging and spectroscopy of biological tissue, for example, arterial plaques or blood clots. A coil according to the teachings of the invention can be incorporated into a flexible catheter that can navigate through a patient's artery to place the coil in proximity of the biological tissue to be imaged. The coil can generate magnetic fields, in response a current flow therethrough, that can excite selected nuclear spins within the interest, and can detect signals generated by the spins in response to the excitation. Alterntively, another coil, e.g., a coil in a magnetic resonance scanner, can be employed to excite the nuclear spins, and the intravascular coil can be utilized to detect signals generated by the spins in response to the excitation.

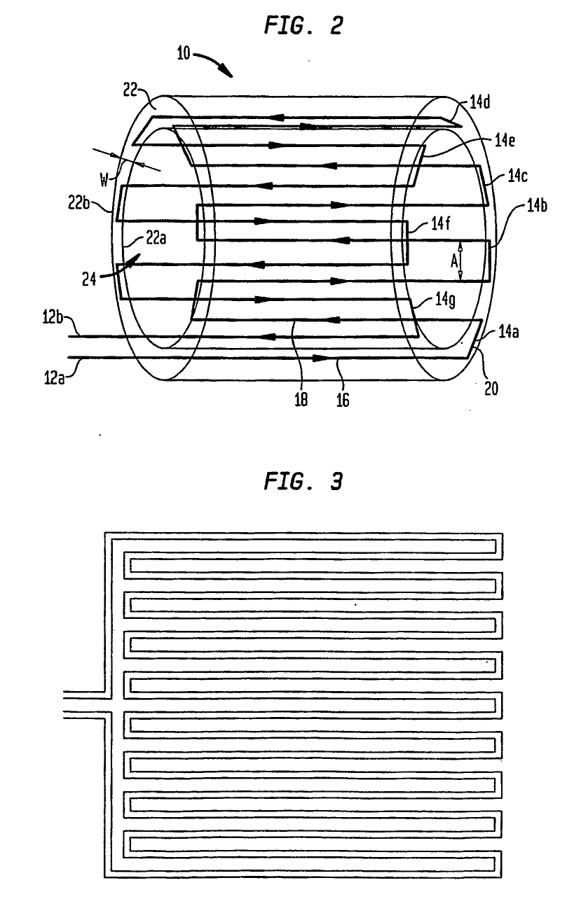

FIG. 2 schematically illustrates an exemplary cylindrical meanderline coil 10 according to the teachings of the invention that can be utilized for exciting and/or receiving signals from a collection of spins in MR imaging or spectroscopy, and in particular in MR spectroscopy and imaging of biological tissue, such as arterial plaques or blood clots. The exemplary coil 10, which has a meanderline structure exhibiting a substantially cylindrical profile, includes two terminals 12a and 12b that function as input and output leads for a current flowing through the coil 10. The exemplary coil 10 is formed of a plurality of conductive segments 14a, 14b, 14c, 14d, 14e, 14f, and 14g, herein referred to collectively as conductive segments 14, that form the substantially cylindrical profile. Each segment 14 includes a pair of elongated conductors, e.g., conductor 16 and 18 that are electrically connected via a bridging conductor 20, that are spaced apart by a selected distance A from one another. Although in this exemplary embodiment, the spacing between the conductor pairs of different conductive segments is uniform, in other embodiments non-uniform spacing can be employed. The elongated conductors of each conductive segment are disposed parallel to one another such that the direction of a current flowing in the coil 10 through one conductor of the pair, e.g., conductor 16, is opposite to the direction of the current through the opposed conductor of the pair, e.g., conductor 18. In this manner, a non-vanishing magnetic field is generated in a substantially annular region surrounding the conductive segments while the magnetic field strength outside the annular region is very low. For example, the magnetic field strength in a region within the cylinder beyond the annular region surrounding the conductive segments is less than the average magnetic field strength in the annular region by about 20 to 60 dB. Most preferarbly, the magnetic field completely vanishes within the cylinder in regions outside the annular region.

More particularly, the exemplary cylindrical meanderline coil 10 generates a non-vanishing magnetic field distribution in an annular region 22 spanned about the conductive segments 14. The exemplary annular region 22 has a width W that is of the order of the spacing A between the conductive pairs of the conductive segments 14. The magnitude of the magnetic field generated by the coil 10 within the annular region 22 decreases from a maximum value at locations in proximity of the conductive segments 14 to very low values at boundaries 22a and 22b of the annular region 22. In particular, the cylindrical meanderline coil 10 advantageously generates substantially vanishing magnetic fields in an inner portion 24 of the cylindrical profile formed by the conductive segments 14. As discussed below, when the coil 10 is employed intravenously, this magnetic field distribution advantageously allows applying a magnetic field to a plaque or a blood clot formed on an arterial wall without exciting spins in the blood flowing through the inner portion of the coil's cylindrical profile, and additionally allows the detection of magnetic resonance (MR) signals from excited spins of a plaque or a blood clot in an arterial wall while minimizing, or preferably eliminating, detection of interfering signals from the blood flowing through the inner portion of the coil's cylindrical profile and/or from tissue far from the arterial wall.

A variety of manufacturing techniques, such as photolithography, can be utilized for constructing a cylindrical meanderline coil of the invention. For example, FIG. 3 schematically illustrates a negative photolithography mask that can be used to etch a 9 loop meanderline coil in copper-clad flexible printed circuit material that can be rolled into a cylinder to form a cylindrical meanderline coil of the invention. Alternatively, the conductors can be formed from wires or electrically conductive tapes or by electrodeposition of conductive material onto an insulating substrate. Further, the coil's conductors can be insulated from an external environment by utilizing flexible or rigid insulating materials.

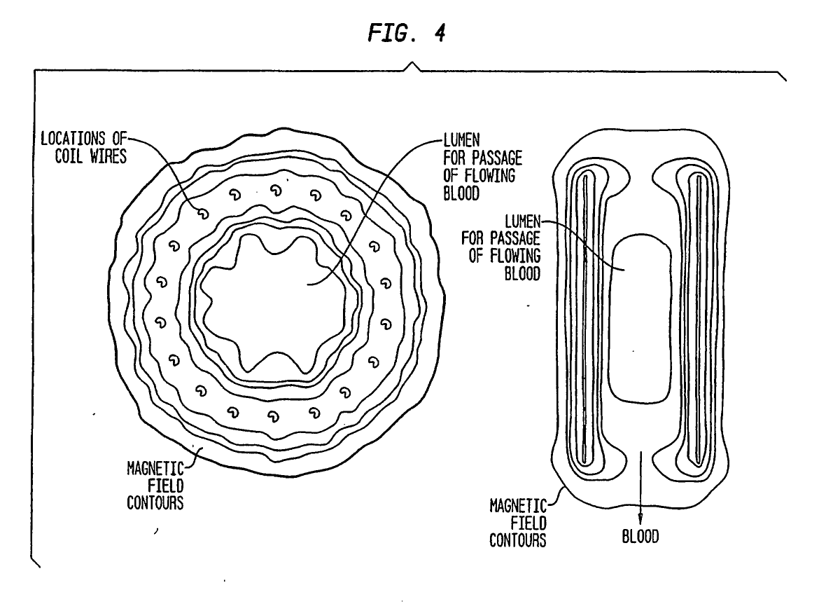

FIG. 4 schematically illustrates the results of numerical simulations illustrating transverse and axial views of the magnetic field distribution associated with a cylindrical meanderline coil according to the teachings of the invention. Both the illustrated transverse and axial views indicate that the generated magnetic field distribution exhibits non-vanishing values in an annular region and exhibits substantially vanishing values within the cylindrical profile of the coil, particularly in the central region of the cylinder.

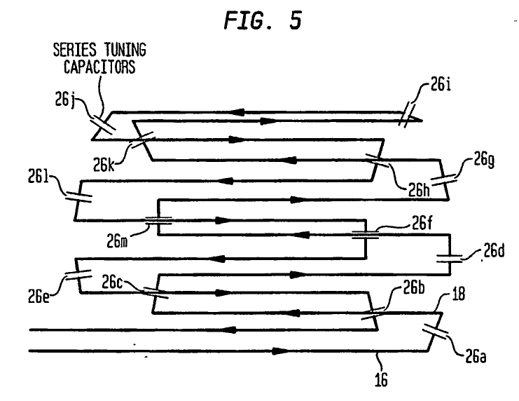

A pair of capacitors can be connected at the leads 12a and 12b of the coil 10 to allow tuning the coil to a selected frequency, and/or matching the coil's impedance to the impedance of other components needed for applying a signal to the coil and/or processing a signal received by the coil. Alternatively, a plurality of capacitors can be distributed along the coil 10 for such tuning and impedance matching. By way of example only, as shown in FIG. 5, a plurality of capacitors 26a-26m, herein collectively referred to as capacitors 26, can be distributed along the coil 10 such that each capacitor connects two parallel conductors of each conductive segment 14. For example, the capacitor 26a electrically connects the conductor 16 to the conductor 18. These capacitors effectively function as distributed series tuning capacitors. Those having ordinary skill in the art will appreciate that other arrangements of capacitors distributed along the coil 10 can be employed for obtaining a desired capacitance.

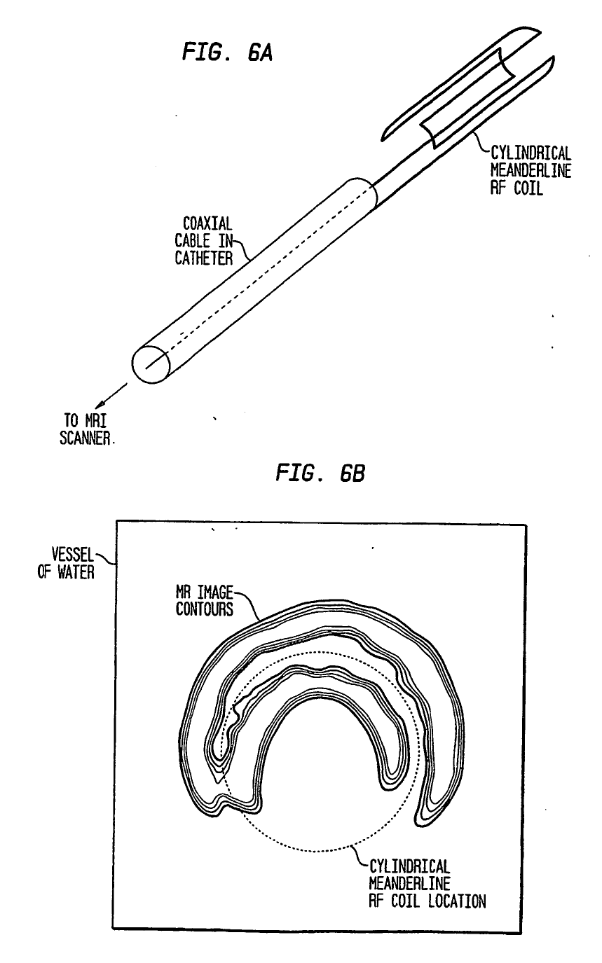

By way of example, and only to illustrate the feasibility of constructing and utilizing a cylindrical meanderline coil according to the teachings of the invention, FIG. 6A illustrates a cylindrical meanderline coil according to the teachings of the invention formed of six conductors and having a 3 mm cross-sectional diameter, and FIG. 6B shows an MR image of water obtained by this coil by immersing it in water and using the coil as both a transmitter to excite the proton spins in the water and also as a receiver to detect the MR signal from the excited spins.

In FIG. 6B, the two bright regions 11 and 13 correspond to the excited and detected spins in the water. The dark ring between the two bright regions marks the cross section of a cylindrically rolled up flexible printed circuit that carries the conductors of the coil. The rolled up printed circuit is coated with an insulating material to insulate the conductors of the coil from direct electrical contact with the water. Because the printed circuit and insulating materials contain no water, their cross section is dark in the image. The image in FIG. 6B clearly shows the excellent localization of the sensitive region, i.e., the region having a non-vanishing magnetic field distribution, of the cylindrical meanderline coil to a substantially annular volume containing the conductors of the coil. The fact that the bright areas are not completely circular is understood to be due to electrical asymmetries in the coil and its parasitic capacitive coupling to the water in which it is immersed. Additional numerical simulations of the type illustrated in FIG. 4 but with the parasitic capacitive coupling included in the simulation yield an asymmetric distribution of RF magnetic field that closely matches the experimental image in FIG. 6B. Further simulations provide evidence that the asymmetric distribution of RF magnetic field may be partially or substantially corrected with the use of distributed tuning capacitance. It should be understood that the coil depicted in FIG. 6A and the data of FIG. 6B are presented only for illustrative purposes, and are not intended to provide necessarily optical embodiment of a coil of the invention, nor optimal magnetic field distributions that can be obtained by employing a coil constructed according to the teachings of the invention.

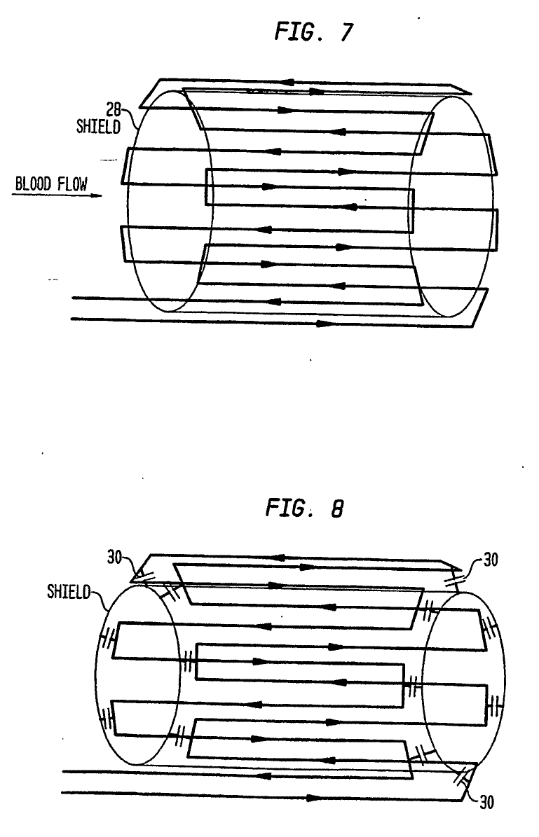

As shown in FIG. 7, in some embodiments of the invention, a substantially cylindrical shield 28 that substantially conforms to the cylindrical profile of the coil 10 and is preferably formed of a conductive material, e.g., copper, can be disposed coaxially within the coil 10 to act as a radiofrequency shield to further diminish, and preferably eliminate, any magnetic field present in the inner portion of the coil 10, thereby better defining an excitation volume associated with the coil 10. The shield 28 can be formed as a solid cylindrical sheet, or as a plurality of conductive segments serving to act as a shield at the RF frequency, while suppressing low frequency eddy currents induced by magnetic field gradient switching of a scanner. Other structures for shielding radiofrequency radiation suitable for use in the practice of the invention are readily apparent to those having ordinary skill in the art.

In embodiments in which a shield is utilized, the distributed capacitance between the coil and the shield can form a part of the tuning and impedance matching circuitry. For example, some degree of tuning and impedance matching of the coil 10 can be obtained by varying the distance between the coil's conductive segments and the shield. Further, as shown ischematically in FIG. 8, a plurality of capacitors 30 connecting the bridging conductors of the coil's conductive segments to the shield may also be employed for adjusting the distributed capacitance of the coil-shield system. Those having ordinary skill in the art will appreciate that other arrangements of capacitors can be employed to obtain desired tuning and impedance matching characteristics of the coil. By utilizing multiple distributed tuning capacitors, a cylindrical meanderline coil of the invention can approximate a transmission line with continuously distributed inductance and capacitance.

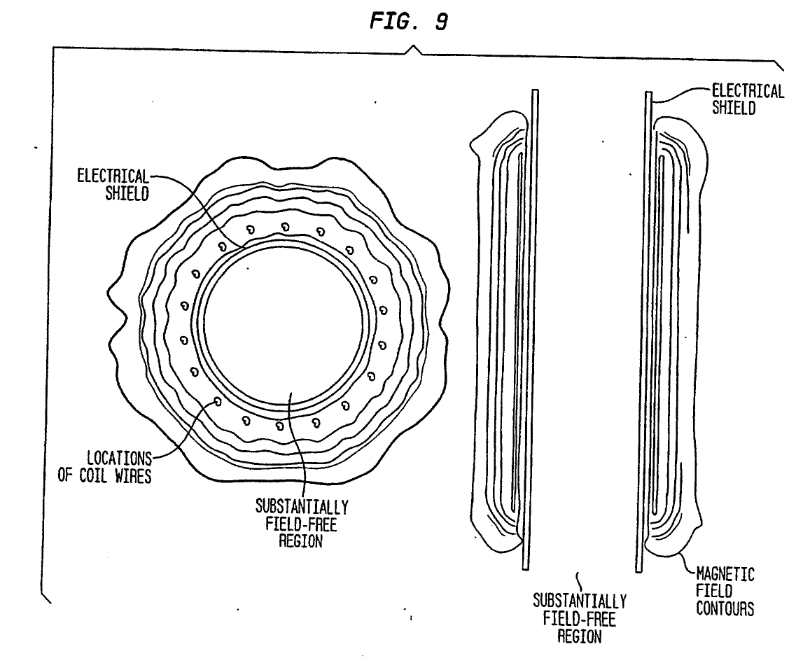

FIG. 9 schematically illustrates the results of a theoretical simulation of magnetic fields generated by a cylindrical meanderline coil of the invention having an RF shield. The illustrated transverse and axial views of the simulated magnetic fields indicate that the fields have vanishing values within the inner portion of the shield. Further, the non-vanishing magnetic field distribution outside the shield exhibits a continuous decrease in magnitude with increase in distance from the conductive segments of the coil. Comparison of FIG. 9 with FIG. 5 illustrates that the shield substantially reduces, and preferably eliminates, penetration of magnetic field generated by the coil into the inner portion of the cylindrical profile.

In other aspects of the invention, coils having meanderline structures, e.g., flat coils or cylindrical coils, are employed for RF quadrature operation. In quadrature operation, the RF coil can be driven by two substantially equal amplitude sources which have a relative phase shift of substantially 90 degrees, producing a circularly polarized RF magnetic field, rather than the linearly polarized RF field in a singlature (non-quadrature) coil. This provides significant advantages in enhancing transmit power efficiency. In addition, the signal-to-noise ratio can be enhanced when a quadrature coil is used to receive the nuclear signal.

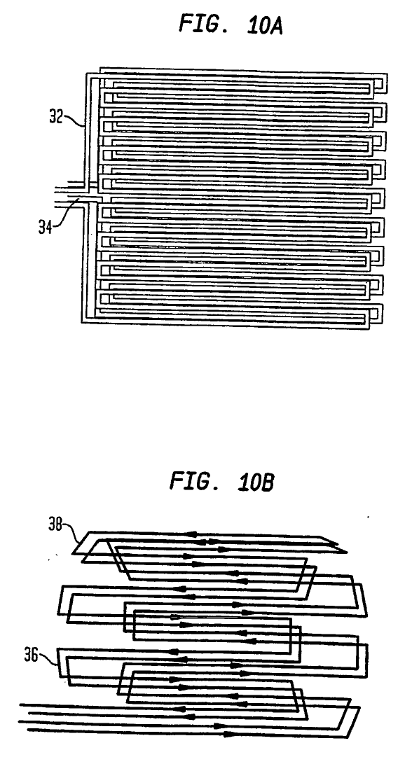

By way of example, FIG. 10A schematically illustrates two flat meanderline coils 32 and 34 that can be utilized for quadrature operation. The coils 32 and 34 can be offset from one another by a selected distance chosen to minimize inductive coupling therebetween, and they can be driven by signals having a 90 degree phase shift relative to one another. In another embodiment shown schematically in FIG. 10B, a pair of cylindrical meanderline coils 36 and 38 according to the teachings of the invention are disposed coaxially relative to one another to form a coil arrangement suitable for RF quadrature operation. The coil 36 can be rotated relative to the coils 38 about the axial direction by a selected angle so as to minimize inductive coupling between the coils.

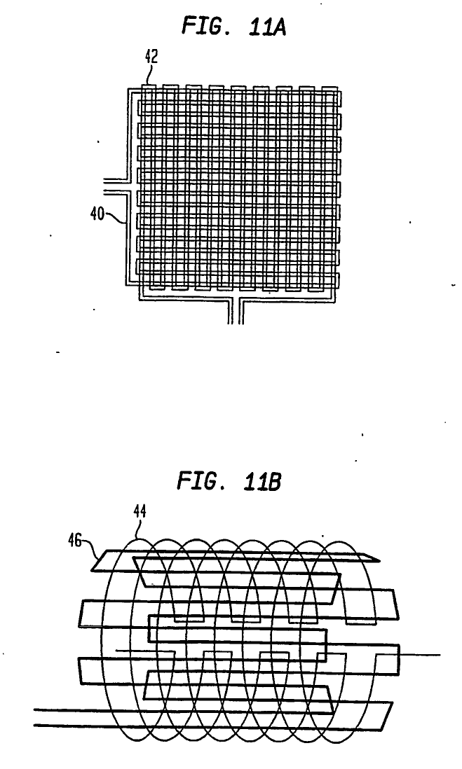

The coil arrangements according to the teachings of the invention for quadrature operation are not limited to those described above. For example, FIG. 11A schematically illustrates a pair of nearly overlapping flat meanderline coils 40 and 42 disposed at 90 degrees relative to one another, i.e., a conductor in one coil is substantially orthogonal to a corresponding conductor in the other coil, to form a coil arrangement suitable for quadrature operation. As another example, FIG. 11B illustrates two nearly overlapping cylindrical meanderline coils 44 and 46 that are coaxially disposed relative to one another. The conductors forming the coaxial coil 44 are oriented at substantially 90 degrees relative to the conductors forming the coil 46 in order to minimize conductive coupling between the coils. The coils 44 and 46 can be driven by RF signals having a 90 degree phase shift relative to one another for quadrature operation. A variety of other coil arrangements in accordance with the teachings of the invention can also be utilized for quadrature operation.

The coil arrangements of the invention suitable for quadrature operation, such as the exemplary coil arrangements described above, can be utilized for performing double resonance MR measurements and/or for tailoring the distribution of the generated magnetic field to obtain a desired geometry of the sensitive volume. In double resonance MR measurements, two, or more in multiple resonance measurements, nuclear spin systems are excited at their different Larmor frequencies, requiring the coil assembly to be resonant at both frequencies. This can be achieved with separate, independently tuned coils covering a shared volume, or with a single coil connecting to a tuning circuit with two input ports such that the single coil is a shared inductive element among the input ports. In some double resonance designs using two coils, it is helpful to minimize the mutual capacitive and/or inductive coupling of the two coils. One example of a double resonance cylindrical meanderline coil using two component coils with minimal mutual inductance is the combination of two nearly overlapping such coils, with their wires oriented at 90 degrees relative to one another, and driven by the different frequency RF sources. Those having ordinary skill in the art will appreciate that many other configurations within the scope of the invention can be employed for double resonance operation.

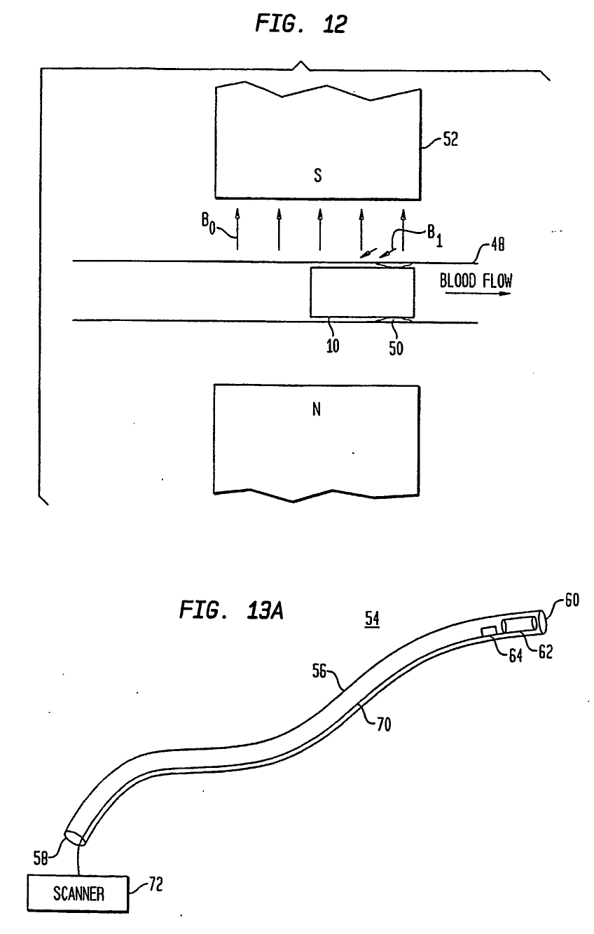

A cylindrical meanderline coil of the invention, such as the above exemplary coil 10 with or without a shield, can find a variety of applications for performing MR imaging or spectroscopy. For example, a coil of the invention can be employed for MR imaging or spectroscopy of plaques or blood clots on arterial walls. For example, with reference to FIG. 12, a cylindrical meanderline coil 10 of the invention can be disposed in a patient's artery 48 at a desired location, e.g., in proximity of a plaque 50. The coil can be selectively positioned in the patient's artery, for example, by utilizing a catheter constructed according to another aspect of the invention described in detail below. A permanent magnet 52 can generate a static magnetic field B₀ for polarizing a selected collection of spins, for example, protons or phosphorous nuclei, of the plaque. The coil 10 can be tuned to the Larmor frequency of the selected nuclear spins of the plaque, and can excite these nuclear spins by application of a magnetic field B₁ thereto. The signals generated by the exited nuclear spins can be detected by the coil and transmitted to other components (not shown) for amplification and analysis. One advantage of the coil 10 is that the magnetic fields within the coil have vanishing values, especially if a shield is employed. Thus, the excitation and reception of signals from blood constituents flowing through the inner portion of the cylindrical coil are substantially diminished, and preferably eliminated. In addition, the magnetic fields generated by the coil that penetrate the arterial wall have defined spatial extensions, e.g., they fall off with distance from the coil, and hence allow localizing excitation and reception signals to selected portions of the arterial wall. Moreoever, a cylindrical meanderline coil of the invention advantageously exhibits enhanced filling factor for intravasculcar imaging or spectroscopy of biological tissue in a vessel wall, and reduced filling factor for blood flowing through the vessel.

The conductors in a coil of the invention can be oriented at any angle with respect to B₀, although there will typically be a variation in performance of the coil as the angle is varied. Other related configurations of the conductors of the meanderline coil are within the scope of the invention. For example, a meanderline structure shaped as an incomplete cylinder, for example, a sector of a cylinder, is within the scope of the invention. Similarly, a warped surface containing a meanderline structure, such as that required to conform to the shoulder or skull of a person, e.g., a helmet shape, is also within the scope of the invention. Additionally, the RF coil can be rigid or flexible. In another embodiment of the invention, the conductors of the RF coil can be twisted about the cylindrical axis into a spiral form rather than being straight.

In another aspect, the invention provides an intravascular flexible catheter that includes a coil at its tip for exciting and/or receiving signals from a collection of spins. A miniature preamplifier is coupled to the catheter in proximity of the coil for amplifying signals applied to or received from the coil. The proximity of the preamplifier to the coil substantially reduces signal degradation that would otherwise occur if long transmission lines were employed to transmit signals between the coil and a preamplifier disposed at a substantial distance from the coil. By way of example, FIG. 13A schematically illustrates an intravascular catheter 54 according to the teachings of the invention that includes a flexible body 56 that extends from a proximal end 58 to a distal end 60. The flexible body is preferably formed of a biocompatible material, for example, polyurethane, and is sized so as to allow its insertion and navigation through a patient's artery. An RF coil 62 is coupled to the catheter body in proximity of its distal end. The coil 62 can have a variety of structures. For example, it can have the cylindrical meanderline structure of the exemplary coil 10 described above. The coil 62 can be utilized, for example, to excite a collection of spins in a biological tissue, and/or to receive magnetic signals from the excited spins. For example, the coil 62 can be employed for detecting magnetic resonance signals from atherosclerotic plaques in arterial walls. In such an application, the dimensions of the coil are preferably selected to be comparable to the dimensions of the plaque to provide an enhanced filling factor. The coil 62 is preferably sufficiently small to be readily inserted into a patient's artery via the catheter. Moreover, the coil and its associated tuning circuitry, e.g., capacitors, inductors, are properly electrically insulated from the patient's body fluids, e.g., blood.

With continued reference to FIG. 13A, a miniature preamplifier 64 is coupled to the catheter body in proximity of the coil 62. The preamplifier 64 is preferably placed as close as possible to the coil 62, and is electrically connected thereto in order to amplify magnetic resonance signals detected by the coil 62. In addition to improving signal to noise ratio, placing the amplifier in proximity, and preferably directly at the coil, can also alleviate problems associated with impedance matching and decoupling of RF coils from one another.

In some preferred embodiments, the preamplifier 64 has dimensions of the order of a few millimeters, and preferably, one to about 2 millimeters, and can withstand exposure to intense RF and magnetic field gradient pulses from a magnetic resonance imager. The catheter's preamplifier needs to be designed so as to occupy minimum space and to be substantially unaffected by magnetic fields and proximity to tissue. Hence, in many embodiments, ferrite cores and most wound inductors, electrolytic capacitors and transformers are not employed in construction of the preamplifier. Further, a standard duplexer circuit that switches the coil between transmit and receive conditions, often employing a quarter wavelength cable, is typically replaced with compact lumped element circuitry. In some embodiments, low noise gallium arsenide field effect transistor (GaAsFET) circuits, often employed in high performance narrow band preamplifier applications, are utilized for the construction of the preamplifier 64.

In some embodiments, an unpackaged transistor on a semiconductor die, together with other circuit elements such as microminiature capacitors, are utilized for constructing the preamplifier 64. Proper insulation and packaging of the entire circuit must be employed to ensure that the preamplifier can function safely in proximity of biological tissue, and to allow integration within the catheter body. Alternatively, the entire preamplifier circuitry can be fabricated as a single chip microcircuit, further reducing the size and eliminating the need for separate bulky external circuit elements. Moreover, as discussed in more detail below, a DC power input to the preamplifier can share the same cable utilized to transmit a detected MR signal out of the patient's body. In other embodiments, the need for a cable electrical connection to the coil and the preamplifier can be eliminated by utilizing inductive or electromagnetic (radio) coupling directly through the patient's body (telemetry) to supply power to and extract signals from the circuit.

With reference to FIG. 13B, in some embodiments, an electronics package 65 electrically coupled to the coil 62 is housed within the catheter 56 at a distance of a few centimeters or more from the coil, rather than being in contact or in close proximity thereof. The electronics package 65 can include electronic devices, such as, pre-amplifiers, varactor diodes, etc, that may be needed for applying excitation signals to the coil and/or amplify signals detected by the coil. This allows selecting the size of the portion of the catheter that houses the coil to be sufficiently small, e.g., having a diameter of 2 mm or less, so as to readily navigate through small vessels, e.g., a small coronary artery. The portion of the catheter that houses the electronic package can be larger in size, e.g., having a larger cross-sectional diameter, to accommodate the electronic package, which may be too large to fit in the other portion. Because vessels narrow progressively, the wider section of the catheter that houses the electronic package can be positioned in the wider portion of a vessel of interest, or in a chamber connected to the vessel (e.g., in the left atrium of the heart when imaging a small coronary artery), while navigating the narrower portion containing the coil into the narrow section of the vessels for performing spectroscopy and/or imaging. This small separation of the electronics package from the coil does not substantially degrade the performance of the coil and the electronics package, and is far superior to placing the electronics completely outside the patient's body. An additional advantage of separating the electronics package from the coil is that, because the intravascular coil and the electronics package are generally inflexible, the flexibility of the entire catheter is increased. This enhanced flexibility is advantageous when inserting and positioning the catheter within the circulatory system.

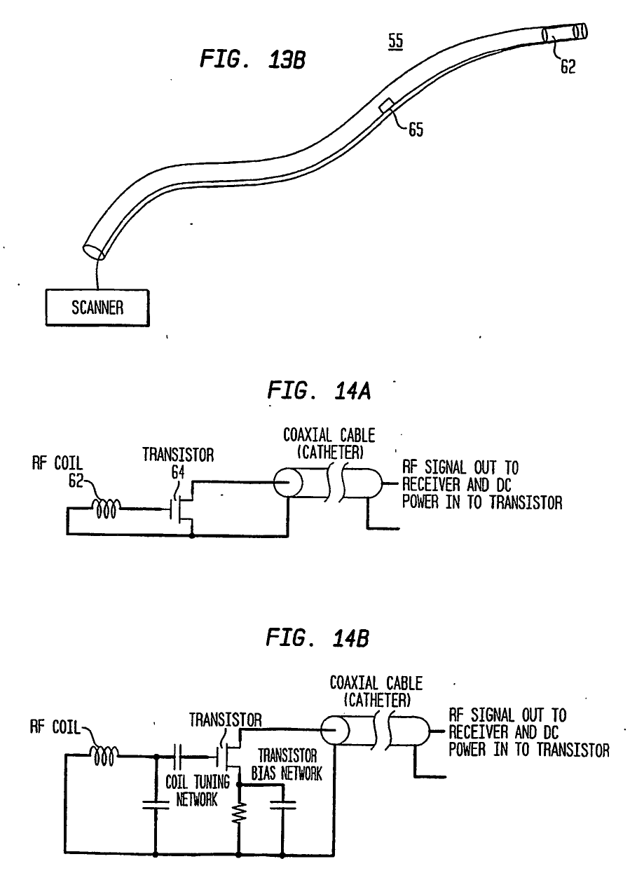

By way of example, FIG. 14A depicts an exemplary electrical circuit for a combined RF coil/preamplifier according to the teachings of the invention coupled to a catheter's tip. A FET transistor 64 coupled to the RF coil is utilized to amplify signals detected by the coil, and conductors 66 and 68 are employed to transmit the RF signals detected by the coil to a receiver, and to provide the transistor with DC power. Further, FIG. 14B depicts exemplary coil tuning/transistor input coupling circuitry and transistor bias circuitry that can be incorporated in the coil/preamplifier circuit of FIG. 14A.

Referring again to FIG. 13A, in the catheter 54, a conductive cable 70, which runs from the distal end to the proximal end of the catheter body, is employed for applying signals to the coil and transmitting signals detected by the coil to external circuit elements, for example, a magnetic resonance scanner 72, for amplification and processing. The cable is typically a transmission line with electrical conductors formed of a conductive material, for example, copper or silver, and has a sufficiently small diameter to allow its positioning within the catheter body. In this embodiment, a single cable is employed for providing DC power to the preamplifier, and to transmit signals detected by the coil to external circuitry. A variety of external excitation and control circuitry can be employed for exciting the nucleir and processing signals detected by the coil. By way of example only, in this embodiment, the scanner 72 can include a power supply that can apply power to the preamplifier. The DC power to the preamplifier can share the cable 70 with the preamplified MR signal that goes back to the detection and processing circuitry of the scanner. The exemplary detection and processing circuitry can include, for example, an amplifier for further amplification of the detected signal, and an analyzer for processing the amplified signal.

As discussed briefly above, placing the preamplifier 64 in proximity of the coil 62 provides a number of advantages. For example, it enhances the signal to noise ratio of the detected signal. In general, for small well designed RF coils used in scanning biological tissues (keeping other factors fixed), the ultimate signal to noise ratio of the scanner is largely determined by the preamplifier signal to noise ratio, usually expressed as the noise figure NF (defined as the ratio in decibels of the amplifier's equivalent input noise power to the inherent noise power emitted by an ideal perfectly impedance matched input impedance). In contrast, for coils of large volume receiving signals from biological tissue, and a properly designed receiver, the tissue is the dominant noise source. Because no amplifier is perfect, every physically real amplifier has a noise figure greater than 0 dB, and therefore adds some noise to any signal that it amplifies. Similarly, every passive electrical component, such as a resistor, has an inherent noise power P, which for an ideal resistor of resistance R ohms is given by P=4 kTB watts, where k=1.384×10⁻²³ J K⁻¹ is Boltzmann's constant, T is the absolute temperature in Kelvins (K), and B is the bandwidth in Hz. The mean square noise voltage across the resistor is <V²>=4 kTBR. This noise arises from the random motions of electrical charge carriers (electrons), and is inherent in any physically real device.

The RF coil has an equivalent resistance, and therefore a corresponding inherent noise power. Similarly, the subject, being composed of biological tissues having some finite electrical conductivity, also has an inherent noise power. Although for large volume coils, the tissue noise usually dominates the coil noise, for small RF coils, the coil noise is likely to dominate the noise from the tissue. Similarly, for small RF coils optimally coupled to the preamplifier, the noise added by the preamplifier will be either dominant or, for the very lowest noise preamplifers, will be the primary source of noise of a well designed receiver. Any attenuation suffered by the signal prior to preamplification results in a reduction in signal to noise ratio because the signal has been attenuated, while the system noise is fixed by the preamplifier and coil. It is therefore important that in any well designed MR receiver: a) the coil circuit is optimally tuned so as to maximize the coupling between the oscillatory magnetic field of the spins and the electrical power output (that is, the coil is tuned and impedance matched); b) there is minimum attenuation between the tuned coil and the preamplifier; c) the preamplifier has the lowest possible noise figure; and d) the preamplifier has sufficient power gain so as to overcome any subsequent attenuation prior to the next stage of amplification, as well as any noise added by the next stage amplifier. Once these conditions are met, the system signal to noise ratio is substantially fixed, and the signal may be subjected to significant losses in the subsequent circuitry without degrading the system signal to noise ratio. The demands on the noise performance of the second stage amplifier are considerably relaxed compared to those on the preamplifier.

Although in the above exemplary catheter 54, the cable 70 can introduce some signal loss, the proximity of the preamplifier to the coil allows substantially boosting the signal intensity by the preamplifier prior to signal transmission through the cable, and compensating for cable losses between the preamplifier and the scanner by additional amplification stages in the scanner, if necessary.

In absence of amplification of the signal detected by the coil prior to its transmission through the cable 70, the noise introduced by the cable may degrade the signal to noise intensity to such an extent that no amplification would recover the signal from the noise. This is particularly true when the cross-section of the cable 70 is small. Because of the miniscule cross section of the conductors of a cable 70 that is suitable for human arteries, a small diameter coaxial cable that is usually used to connect a catheter RF coil to the scanner typically has extraordinarily high attenuation compared to the cable normally used for scanner interconnections, in some cases as high as 100 dB per including the deleterious effect of severe impedance mismatches) if the coil were properly tuned and perfectly impedance matched to the characteristic impedance of the cable. In this case, the corresponding loss in signal to noise ratio is numerically equal to the attenuation. However, difficulties in perfectly tuning and impedance matching the coil can dramatically magnify the cable losses. It is therefore possible for actual signal losses to amount to upwards of 10-20 dB. This is equivalent to a factor of on the order of 3-10 in signal to noise ratio, corresponding to a factor on the order of 10-100 in signal averaging time to recover the signal to noise ratio lost by the cable attenuation.

Placing a simple low noise preamplifier at or near the catheter coil substantially eliminates the severe degradation in system noise figure introduced by the catheter transmission line. As long as this preamplifier's noise figure is low (for example, 0.5 dB), and its gain is sufficient to overcome the cable losses (for example, 25 dB), the inherent receiver system noise figure is preserved. These values are typical of the low noise gallium arsenide field effect transistor (GaAsFET) circuits often used in high performance narrowband preamplifier applications. Specifically optimized preamplifier circuits can achieve even higher performance values. Additional advantages of placing a preamplifier near the coil include the ability to impedance match the coil directly to the preamplifier input impedance, which can yield an optimal noise figure (typically different from the 50 ohm characteristic impedance of the cable), and to design an amplifier input impedance which permits effective decoupling of the catheter RF coil from other RF coils in the scanner.

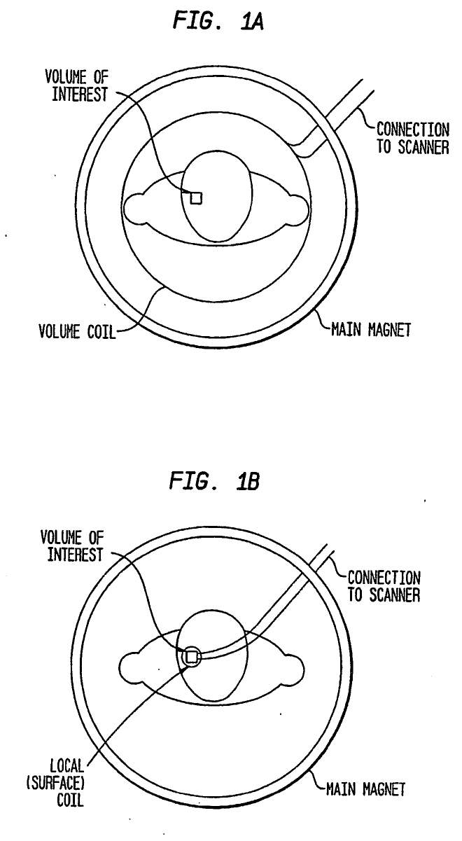

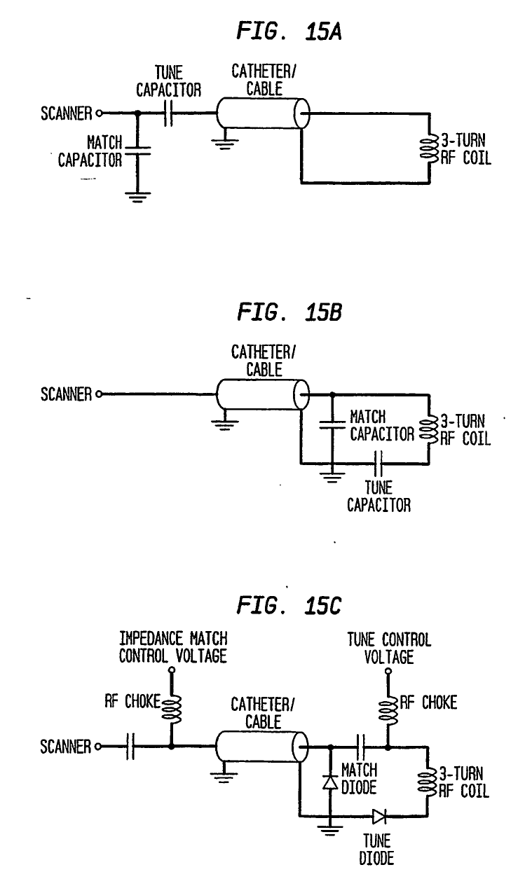

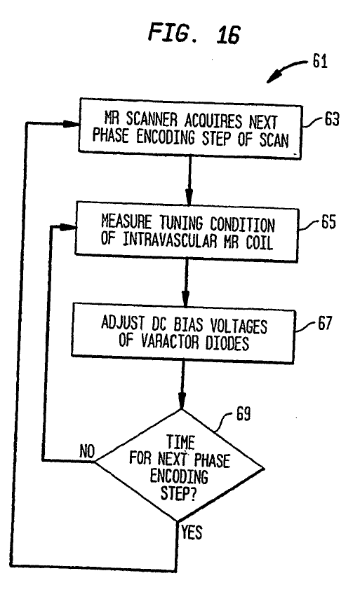

Although other electronic elements needed, for example, for tuning the coil and/or impedance matching, can be disposed external of the catheter, as shown in FIG. 15A, in some preferred embodiments of the invention, these components, e.g., matching and tuning capacitors, are housed within the catheter, and preferably in proximity of the coil and the preamplifier. For example, FIG. 1 SB schematically illustrates that a matching capacitor and a tuning capacitor can be coupled to the RF coil within the catheter to provide tuning and impedance matching.

In some embodiments, varactor diodes are employed to provide adjustable capacitance whose value can be controlled by external DC voltages. PIN diode switches required for transmit/receive switching, selecting among multiple coils or multiple operating frequencies, or protecting the preamplifier from RF pulses, may also be incorporated within the catheter. Much of this circuitry can be integrated onto one or two integrated circuits in order to achieve ultra compact circuitry. As discussed above, gallium arsenide can be utilized for constructing the preamplifier transistor. The other active electronic functions may be combined on the gallium arsenide chip (or a hybrid gallium arsenide/silicon device), or may be implemented on a separate silicon integrated circuit.

It is to be understood that the schematic diagrams of FIGS. 14 and 15 are exemplary only, and may require additional electronic components, readily known to those having ordinary skill in the art, to be fully functional circuits.

By way of example, FIG. 15C illustrates an exemplary circuit diagram of a tuning circuitry for a coil in a catheter of the invention in which varactor diodes are employed to adjust the impedance transformation of the coil to either the coaxial cable as illustrated, or to a preamplifier. Because an intravascular coil placed in a blood vessel is in constant motion due to heart beat, breathing, pulsatile flow of blood, and other voluntary and involuntary motions of the subject, the tuning condition of the coil is constantly changing during the scan. The tuning condition may also change as the catheter containing the coil is advanced through a vessel or pulled back by a physician performing the medical procedure. Therefore, an important use of electronically remote tuning of an intravascular coil of the invention with varactor diodes is to maintain continuous optimal adjustment of the tuning capacitors. The adjustment of the tuning by setting the DC bias voltages of the varactor diodes may be performed manually, e.g., by a human operator, or automatically by an analog or digital circuit, including a computer.

In a preferred embodiment of the tuning procedure, the tuning condition of the intravascular coil is sensed briefly in the time interval between phase encoding steps of an MR scan (for example by exciting the coil with a weak pulse of RF power at the scanner operating frequency, and measuring the amplitude and phase of the RF power reflected by the coil). Then the appropriate changes in the DC bias voltages are made to change the capacitances of the varactor diodes such that the reflected power is minimized. In this manner, the intravascular coil is maintained close to, and preferably at, a state of optimal tuning during the MR scan. By way of example, with reference to a flow chart 61 shown in FIG. 16, in a step 63, a next phase encoding step of a scan is acquired by an MR scanner. Subsequently, in step 65, tuning conditions of the intravascular MR coil is measured by utilizing techniques known in the art. In response to these measurements, in step 67, DC bias voltages applied to varactor diodes are adjusted to the optimize the coil's tuning conditions. As shown in step 69, if the time for performing the next phase encoding step has not arrived, the steps 65 and 67 are repeated, otherwise, the above cycle is repeated beginning with the step 63.

Another important use of electronically remote tuning of an intravascular coil with varactor diodes is to detune the coil (for example, to make the coil nonresonant during the pulsing of a separate transmit coil of the scanner) by misadjusting the DC bias voltages.

Although it is simple to multiplex DC power and RF output in a single transmission line (the single cable connecting the coil to the scanner), multiple control signals may alternatively be transmitted with additional very fine wires or cables placed within the same catheter. Alternatively, if integrated circuits are employed, additional circuitry can be utilized to multiplex all signal, power and control functions on a single cable, by digital or other means. Cables for nuclear and control signal and power transmission can be formed of combinations of one or more single unshielded wires, conventional coaxial cables, in which a single conductor is enclosed by a shield, twinaxial cables, in which two balanced conductors are enclosed by a single shield, or triaxial cables in which a coaxial cable is enclosed by a second shield electrically isolated from the coaxial cable shield, or extensions of these configurations. An advantage of triaxial cables is that the outer shield can be arranged to act as a electromagnetic shield to reduce the spurious electrical interaction of its internal coaxial cable with the environment, for example by reducing RF heating of tissue or reducing the tuning sensitivity of the coil and cable

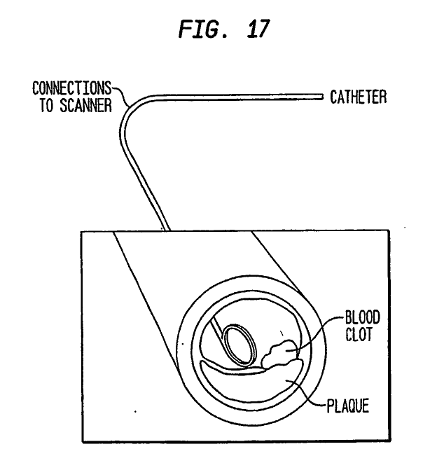

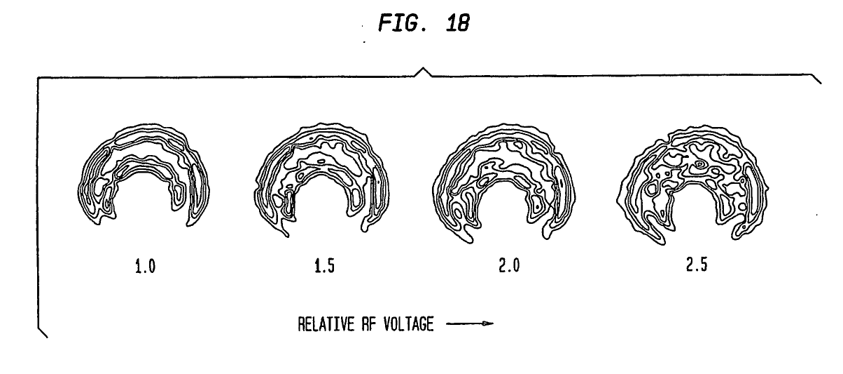

With reference to FIG. 17, in one application of a catheter of the invention, the catheter can be navigated through a patient's artery and positioned in a selected portion of the artery such that the coil is in vicinity of a plaque or a blood clot. The coil can then be employed to apply an excitation magnetic field to a selected collection of spins and to receive signals from the excited spins. Positioning of the coil intravenously, can then be employed to apply an excitation magnetic field to a selected collection of spins and to receive signals from the excited spins. Positioning of the coil intravenously, via the catheter, in proximity of the plaque results in a much higher filling factor and much enhanced signal to noise ratio in comparison with utilizing a surface coil (See FIG. 1B) for obtaining MR spectra and/or images of the plaque.

FIG. 18 shows a series of MR images taken with a 3 mm cylindrical meanderline coil immersed in water. The coil is used as both transmitter and receiver. The image plane is perpendicular to the coil axis. The bright arcs show the annular region of sensitivity in cross section. The dark ring overlapping each arc is due to the fact that the coil itself displaces water, and therefore emits no MR signal. The RF pulse amplitude used to excite the proton spins in the water increases progressively from left to right in the images, covering an overall range of a factor of 2.5. Yet the volume of sensitivity does not increase significantly, demonstrating the tight spatial restriction of the volume of sensitivity characteristic of cylindrical meanderline coils. It should be understood that this exemplary data is presented only for illustrative purposes, and is not intended to necessarily demonstrate an optimal performance of a coil according to the teachings of the invention.

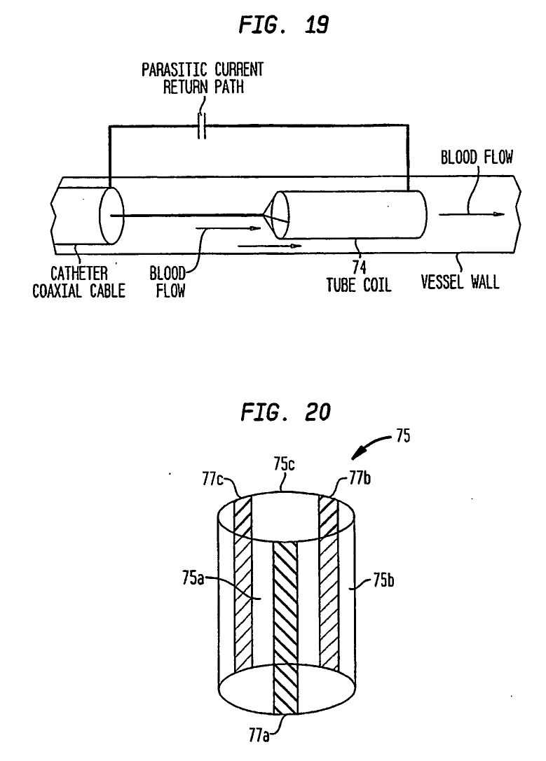

FIG. 19 schematically illustrates another coil 74 according to the teachings of the invention suitable for use in magnetic resonance spectroscopy or imaging. The coil 74, which is herein referred to as “one loop cylindrical meanderline coil” can be constructed as a conductive tube which can be connected at one end to the central conductor of a coaxial cable. There is no wire connecting the other end of the conductive tube to the shield of the coaxial cable. In other words, this is equivalent to replacing the exposed wire of a loopless antenna, with a conductive tube. Similar to the previous meanderline coils of the invention discussed above, when utilized intravascularly, the tubular coil 74 allows free flow of blood therethrough while the RF field generated by the coil 74 is confined to a tubular region proximate to the coil's tubular surface because a current exciting the coil will flow axially along the length of the tube, not around the tube axis. The tubular coil 74 can be manufactured as simply as a loopless antenna, and can be easily miniaturized. In addition, the tubular coil 74 provides all the advantages of cylindrical meanderline coils of the invention described hold it open. In such a case, the current flow would be carefully controlled to be along the tube or the stent axis to gain the advantages associated with cylindrical meanderline coils of the invention. It should be understood that the coil 74 can be also be formed as a portion of a conductive tube.

With reference to FIG. 20, another tubular conductive coil 75 according to the teachings of the invention can be formed of a plurality of conductive segments, such as exemplary segments 75a, 75b, and 75c, each of which is separated and electrically insulated by a dielectric segment, such as segments 77a, 77b, and 77c, from an adjacent conductive segment. Each conductive segment can be utilized individually to detect magnetic signals from a portion of a vessel wall. Alternatively, some or all of the conductive segments can be employed simultaneously.

It is also within the scope of the invention to switch between modes of operation of the intravascular MR coil such that the volume of excitation or sensitivity of the coil is changed, for example, between a first mode of operation in which the coil is sensitive to a long length of an artery and a second mode of operation in which the coil is sensitive to a much shorter length of an artery. As explained previously, an optimal filling factor of an MR coil is achieved when the region of excitation or sensitivity of the coil matches the region of interest to be scanned. Therefore, extended lengths of arteries are preferably scanned with intravascular coils designed for long lengths, whereas short lengths of arteries, for example those containing particular atherosclerotic plaques that a physician wishes to inspect in detail with MR scanning, are best scanned with intravascular coils designed for short lengths. Removing one intravascular coil and replacing it with another takes time, requires that at least two different type of intravascular coils are available, and subjects the patient to additional risk due to the nature of the medical procedure.

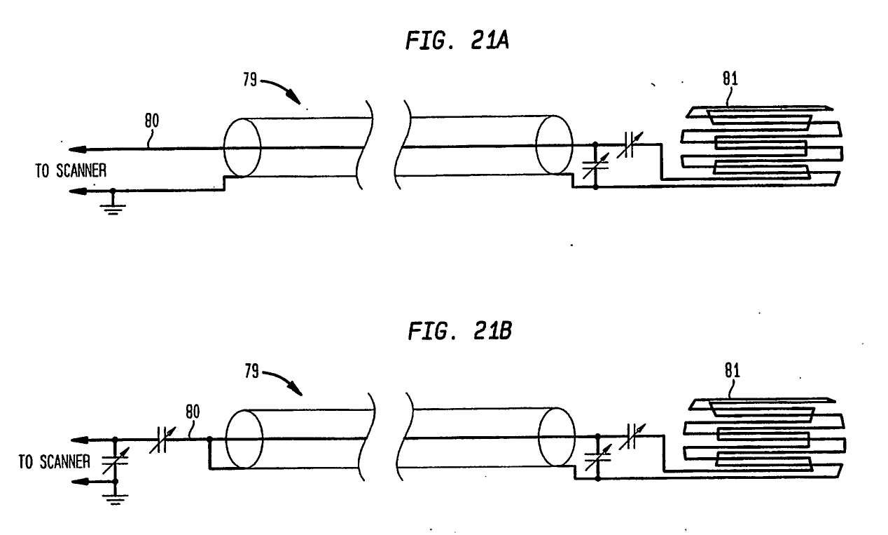

With reference to FIGS. 21A and 21B, one embodiment of the invention provides a catheter 79 that can be operated in two modes, one for imaging an extended length of a vessel of interest, and the other for imaging a smaller portion of the vessel's wall. The exemplary catheter 79 includes an elongated conductor 80, e.g., an inner conductor of a coaxial cable, that extends from a proximal end of the catheter to its distal end. Further, the catheter 79 includes a cylindrical meanderline coil 81, formed in accordance with the teachings of the invention, coupled to its distal end. With reference to FIG. 21B, in one operational mode, the catheter can be connected to an external tuning circuitry that tunes the entire catheter (for example, with the center conductor 80 and the shield of the coaxial cable short circuited together) to resonance as if the entire catheter were a guidewire type of intravascular coil. This allows imaging an extended length of a vessel of interest. With reference to FIG. 21A, subsequent to completion of the extended length scanning, the catheter can be connected to a second tuning circuitry (or directly to a preamplifier of the scanner if the catheter contains tuning circuitry for the intravascular coil at its tip) such that the intravascular coil 81 at the catheter tip defines the volume of sensitivity. This allows imaging tissue disposed in an annular region associated with the coil 81, as previously described, from which magnetic signals can be detected.

The use of a catheter or a coil of the invention is not limited to intravascular spectroscopy and/or imaging. In particular, the coils and the catheters of the invention can be employed for surface NMR spectroscopy and/or imaging of biological tissue. For example, a coil of the invention, for example, in the form of a half cylinder or a helmet, can be employed for imaging the brain cortex. In other applications, a coil of the invention, which can be formed as a flexible structure that can conform to the shape of a body part, can be employed for imaging different body parts, such as the shoulder.

In addition to applications in biomedicine such as blood vessel and gastrointestinal tract wall imaging and spectroscopy, cylindrical meanderline coils of the invention are also suited for conducting measurements in other fields of science, engineering, agriculture and commerce. For example, a cylindrical meanderline coil according to the teachings of the invention can be utilized to selectively acquire MR signals from selected volumes of a fluid, e.g., volumes disposed at a selected distance from an axis of flow, while rejecting other volumes to study, for example, laminar flow in pipes or couette flow around cylinders. In down-hole well logging applications in the petrochemical field, a cylindrical meanderline coil of the invention can be useful in acquiring signal from the surrounding rock while rejecting signal from the drilling mud as an alternative to opposed solenoid MR coil designs.

The cylinder of a meanderline coil of the invention does not necessarily need to have a circular cross-section or to form a complete cylinder. For example, a meanderline coil of the invention can have an elliptical cross-section. Further, flexible meanderline sectors can be formed to conform to curved surfaces, such as the skull (when the cerebral cortex is of interest) or the trunk of a tree, to optimize MR signal collection from such curved geometries. The cylindrical meanderline and its derivative coil designs represent a dramatic gain in data acquisition efficiency because the dimensionality of the measurement is effectively reduced. Rather than collecting spatially resolved data from an entire three-dimensional volume (which is generally highly time consuming) and subsequently selecting only the curved volume of interest, the cylindrical meanderline geometry permits the acquisition of data directly from the desired volume only.

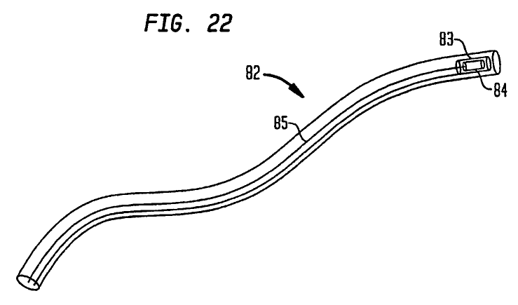

With reference to FIG. 22, another embodiment of the invention provides a catheter 82 having a meanderline cylindrical coil 83, formed in accordance with the teachings of the invention, coupled at a distal end thereof, and a balloon 84 preferably positioned within the coil 83. The balloon 84 has preferably an annular form to allow flow of blood through its inner portion. The balloon can be inflated, for example, via transfer of a fluid, e.g., saline solution, through a lumen 85 extending from the catheter's proximal end to its distal end, to exert pressure on the coil so as to ensure its optimal positing within a vessel and/or its optimal contact with the vessel's wall.

Those having ordinary skill in the art will appreciate that many modifications can be made to the above illustrative embodiments without departing from the scope of the invention.

What is claimed is:

1. A coil for transmitting and/or receiving magnetic excitations, comprising a meanderline conductive structure comprising a plurality of conductive segments forming a substantially cylindrical profile and generating a non-vanishing magnetic field distribution in response to current flow through said coil in a substantially annular region surrounding said conductive segments and a substantially vanishing magnetic field distribution in a region outside said annular region.

2. The coil of claim 1, further comprising an input terminal and an output terminal to allow, respectively, ingress and egress of an electrical current into and out of the coil.

3. The coil of claim 1, wherein each conductive segment comprises a pair of elongated conductors separated by a selected distance and a bridging conductor electrically connecting said pair of conductors such that a flow of current from said input terminal to said output terminal results in current in opposite directions in said pair of conductors.

4. The coil of claim 3, wherein a spacing between the conductor pair of each segment is substantially similar to a corresponding spacing between the conductor pair of another segment.

5. The coil of claim 3, wherein separations between conductor pairs of different conductive segments are non-uniform,

6. The coil of claim 3, wherein said annular region has a width commensurate in size with said selected distance between said pair of conductors.

7. The coil of claim 1, wherein the magnetic field generated by the coil at a location within said annular region decreases as a distance of said location from said conductive segments increases.

8. The coil of claim 2, further comprising at least a capacitor electrically coupled to one of said input or output terminals to allow any of tuning the coil to a selected frequency and matching the coil's impedance to impedance of one or more other components coupled to the coil.

9. The coil of claim 3, further comprising a plurality of capacitors each coupled between said two elongated conductors of one of the conductive segments to function as distributed series tuning capacitors.

10. The coil of claim 1, wherein said conductive segments are formed of copper.

11. The coil of claim 1, further comprising a substantially cylindrical conductive shield disposed coaxially within said coil so as to further diminish said substantially vanishing magnetic field.

12. A coil assembly for radiofrequency quadrature operation, comprising
a pair of conductive coils, each comprising
an input terminal,
an output terminal, and
a plurality of conductive segments extending from said input terminal to said output terminal, each of said conductive segments comprising two elongated conductors disposed substantially parallel to one another such that a flow of current from said input terminal to said output terminal results in opposite current directions in said conductors,
wherein said conductive coils are disposed in proximity of one another such that application of two voltage signals having substantially equal amplitudes and about 90 degree phase difference, each across one of said coils, generates a circularly polarized RF magnetic field.

13. The coil assembly of claim 12, wherein said coils are disposed relative to one another such that the conductors of each conductive segment of one coil are substantially perpendicular to the conductors of a corresponding conductive segment of the other coil.

14. The coil assembly of claim 12, wherein said coils are flat.

15. The coil assembly of claim 12, wherein each of said coils has a cylindrical profile.

16. The coil assembly of claim 15, wherein said coils are disposed coaxially relative to one another.

17. The coil assembly of claim 12, further comprising at least two capacitors each electrically coupled to one of said coils for tuning said coil to a selected frequency.

18. The coil assembly of claim 17, wherein said coils are tuned to different frequencies.

19. A coil for transmitting and/or receiving magnetic excitations, comprising a meanderline conductive structure having an input lead and an output lead, said conductive structure comprising a plurality of conductive segments forming a substantially cylindrical profile, each of said conductive segments comprising at least a pair of elongated conductors disposed substantially parallel to one another such that the flow of current from said input lead to said output lead through the coil results in opposite current directions in each conductor of the pair, thereby generating a non-vanishing magnetic field in a substantially annular region surrounding the conductive segments and a substantially vanishing magnetic field outside said annular region.

20. The coil of claim 19, further comprising a plurality of capacitors disposed along said conductive structure for tuning said coil to a selected frequency.

21. A coil for transmitting and/or receiving radiofrequency radiation, comprising a meanderline conductive structure comprising a plurality of conductive segments collectively forming a selected profile, each conductive segment comprising at least two elongated conductors disposed substantially parallel to one another, said conductive structure further comprising an input terminal and an output terminal such that a flow of current from said input terminal to said output terminal will result in opposite current directions in said two elongated conductors of each of said conductive segments.

22. The coil of claim 21, wherein said selected profile corresponds to a sector of a cylinder.

23. The coil of claim 21, wherein said selected profile conforms to at least a portion of an inner surface of a patient's artery.

24. The coil of claim 21, wherein said selected profile corresponds to a curved surface substantially conforming to a patient's anatomical surface.

25. The coil of claim 21, wherein said meanderline conductive structure is substantially rigid.

26. The coil of claim 21, wherein said meanderline conductive structure is substantially flexible.

27. A medical catheter, comprising
a flexible body extending from a proximal end to a distal end,
a coil coupled to said flexible body in proximity of said distal end for generating and detecting magnetic signals, and
an amplifier coupled to said catheter in proximity of said coil and electrically connected thereto in order to amplify said magnetic signals.

28. The medical catheter of claim 27, wherein said coil comprises a meanderline conductive structure having a plurality of conductive segments forming a substantially cylindrical profile, said conductive segments being configured such that the coil generates, in response to a current flow therethrough, a non-vanishing magnetic field in an annular region in proximity of said conductive segments and a substantially vanishing magnetic field in a region outside said annular region.

29. The medical catheter of claim 27, wherein said flexible body is formed of a biocompatible material.

30. The medical catheter of claim 27, wherein said flexible body is sized to allow navigation of the catheter through a patient's artery.

31. The medical catheter of claim 27, further comprising at least one capacitor electrically coupled to said coil for tuning said coil to a selected frequency.

32. The medical catheter of claim 31, further comprising an inductor electrically coupled to said capacitor and said coil for facilitating tuning said coil to said selected frequency.

33. The medical catheter of claim 32, further comprising an elongated conductor extending from the proximal end of the catheter to its distal end for electrical coupling to said coil, said conductor being capable of transmitting excitation signals from an excitation external circuitry to said coil and/or transmitting signals detected by said coil to a detection external circuitry.

34. The medical catheter of claim 33, wherein said excitation external circuitry comprises a signal generator for applying an excitation signal to said coil for exciting a collection of polarized spins of a plaque disposed on an interior wall of a patient's artery in which the coil is inserted.

35. The medical catheter of claim 34, wherein said detection external circuitry comprises a detector for detecting signals generated by said polarized spins in response to said excitation.

36. The medical catheter of claim 27, wherein said amplifier comprises a low noise transistor.

37. The medical catheter of claim 27, wherein said coil and said amplifier are housed within said catheter.

38. The medical catheter of claim 27, further comprising one or more varactor diodes electrically coupled to said coil and housed within said catheter, said varactor diodes allowing continuous tuning of said coil.

39. The medical catheter of claim 38, further comprising a feedback circuit electrically coupled to said varactor diodes and said coil for periodically monitoring tuning of said coil and adjusting voltages applied to said varactor diodes so as to optimize tuning of said coil.

40. The medical catheter of claim 27, wherein said flexible body includes a first portion at said proximal end having a first cross-sectional size suitable for housing said coil and a second portion having a second cross-sectional size suitable for housing said amplifier.

41. The medical catheter of claim 40, wherein said first cross-sectional size is larger than said second cross-sectional size.

42. A method for magnetic resonance imaging and spectroscopy of at least a portion of a plaque disposed on an interior wall of a patient's artery, comprising
disposing a coil having a substantially cylindrical profile formed of a plurality of conductive segments in the artery in proximity of said plaque, said conductive segments being configured such that a current flow through said coil generates a substantially vanishing magnetic field within a region within said cylindrical profile though which blood flows and a non-vanishing magnetic field in an annular region in proximity of said conductive segments extending into at least a portion of said plaque,
applying a static magnetic field to said plaque to polarize selected atomic nuclei of constituents thereof,
applying a time-varying magnetic field in said annular region in order to excite said polarized nuclei, and
utilizing said coil to detect radiation emitted by said excited nuclei.

43. The method of claim 42, wherein said nuclei are protons.

44. The method of claim 42, wherein said selected nuclei are any of phosphorus, carbon, oxygen, or sodium nuclei.

45. The method of claim 42, wherein said time-varying signal applied to excite the nuclei includes a frequency substantially equal to Larmor frequency of said nuclei.

46. A medical catheter, comprising
a flexible body extending from a proximal end to a distal end,
a coil having a substantially tubular conductive structure for generating and/or detecting magnetic signals, said coil being coupled to said flexible body in proximity of said distal end.

47. The medical catheter of claim 46, wherein said coil generates a magnetic field in proximity of said conductive structure in response to a current flow therein, and substantially vanishing magnetic field in a region at least partially enclosed by said conductive structure.

48. A medical catheter, comprising
a flexible body extending from a proximal end to a distal end and sized for navigation through at least a portion of a subject's circulatory system,
at least one elongated conductor extending along at least a portion of said flexible body, said conductor being adapted for generating and/or receiving magnetic signals during one operational mode of said catheter, and
a coil coupled to said flexible body at a distal end thereof, said coil having a generally cylindrical conductive structure adapted for generating and/or receiving magnetic signals within an annular region surrounding said conductive structure during another operational mode of said catheter.

49. The medical catheter of claim 48, further comprising a first external circuitry coupled to said elongated conductor for tuning said conductor for imaging an extended length of a subject's artery.

50. The medical catheter of claim 49, further comprising a second external circuitry coupled to said coil for tuning said coil for imaging biological tissue within said annular region upon placement of said catheter in a subjet's artery.

51. The medical catheter of claim 50, further comprising a switch coupled to said first and second external circuitry for selecting one of said operational modes of said catheter.
